(19) United States
(12) Patent Application Publication
(10) Pub. No.: US 2017/0259053 A1
(43) Pub. Date: Sep. 14, 2017

(54) PUNCTURE DEVICE

(71) Applicant: KAIWA CO., LTD., Uenohara-shi, Yamanashi (JP)

(72) Inventor: Shigeyuki YAMAZOE, Uenohara-shi, Yamanashi (JP)

(21) Appl. No.: 15/509,486

(22) PCT Filed: Sep. 8, 2015
(86) PCT No.: PCT/JP2015/075453

§ 371 Date: Mar. 7, 2017

(30) Foreign Application Priority Data
Sep. 8, 2014 (JP) 2014-182572
Sep. 7, 2015 (JP) 2015-175878

(51) Int. Cl. A61M 37/00

(52) U.S. Cl. CPC A61M 37/00; A61M 2205/02

(57) ABSTRACT

A device includes a basal portion and a plurality of blade portions disposed on the basal portion. The blade portion has a thin shape, and a distal end portion of the blade portion has a convex shape.

### TECHNICAL FIELD

The present invention relates to a puncture device, and more particularly to a puncture device used for percutaneous administration of a drug.

### BACKGROUND ART

As a puncture device, a microneedle device has been disclosed, which includes a plurality of microneedles for puncturing the skin and allows noninvasive administration of a drug. The microneedle device punctures a horny layer, which is the outermost layer of the skin, with the microneedles to percutaneously administer a drug. Various sizes and shapes of microneedles have been proposed to promote the percutaneous absorption of a drug (for example, patent document 1). Patent document 1 discloses microneedles having a conical shape and a knife-shape. The document also discloses that microneedles can be formed from metal, ceramic, or plastic.

### CITATION LIST

### Patent Document

Patent Document 1: Japanese Translation of PCT International Application Publication No. 2010-502268

### SUMMARY OF THE INVENTION

### Problems to be Solved by the Invention

The microneedle disclosed in the patent document 1 has a fine distal end. When, for example, such a microneedle is formed from plastic, the distal end deforms as it is pressed against the skin. This leads to a failure in puncturing the skin to an extent that allows the administration of a drug.

An object of the present invention is to provide a puncture device which punctures the skin more reliably.

### Means for Solving the Problems

A puncture device according to the present invention includes a basal portion and a plurality of blade portions disposed on the basal portion. The blade portion has a thin shape, and its distal end portion has a convex shape.

### Advantageous Effects of Invention

According to the present invention, the blade portion has a thin shape and hence does not easily deform as compared with a needle-shape. Since the distal end portion of the blade portion has a convex shape, the blade portion easily goes into the skin. This allows the blade portion to cut the skin when a force is applied to the blade portion in one direction by moving the tooth of the needle in its longitudinal direction while the blade portion is pressed against the skin. Therefore, with the blade portions, which cut the skin, the puncture device more reliably punctures the skin.

### BRIEF DESCRIPTION OF THE DRAWINGS

FIG. 1 is a perspective view showing an overall configuration of a puncture device according to a first embodiment.

FIGS. 2A to 2C show a configuration of a blade portion according to the first embodiment: FIG. 2A is a front view; FIG. 2B is a plan view; and FIG. 2C is a side view.

FIGS. 3A to 3C show a configuration of a blade portion according to a second embodiment: FIG. 3A is a front view; FIG. 3B is a plan view; and FIG. 3C is a side view.

FIGS. 4A to 4C show a configuration of a blade portion according to a modification of the second embodiment: FIG. 4A is a front view; FIG. 4B is a plan view; and FIG. 4C is a cross-sectional view cut along a line IVC-IVC in FIG. 4A.

FIGS. 5A to 5C show a configuration of a blade portion according to a third embodiment: FIG. 5A is a front view; FIG. 5B is a plan view; and FIG. 5C is a cross-sectional view cut along a line VC-VC in FIG. 5A;

FIGS. 6A to 6C show a configuration of a blade portion according to a modification of the third embodiment: FIG. 6A is a front view; FIG. 6B is a view taken along an arrow X; and FIG. 6C is a cross-sectional view cut along a line VIC-VIC in FIG. 6A;

FIGS. 7A to 7C show a configuration of a blade portion according to modified embodiment (1): FIG. 7A is a front view; FIG. 7B is a plan view; and FIG. 7C is a side view;

FIGS. 8A to 8C show a configuration of a blade portion according to modified embodiment (2): FIG. 8A is a front view; FIG. 8B is a plan view; and FIG. 8C is a cross-sectional view cut along a line VIIIC-VIIIC in FIG. 8A.

FIGS. 9A to 9C show a configuration of a blade portion according to modified embodiment (3): FIG. 9A is a front view; FIG. 9B is a plan view; and FIG. 9C is a side view.

FIG. 10 is a side view showing a configuration of a blade portion according to modified embodiment (4); and

FIG. 11 is a side view showing a configuration of a blade portion according to modified embodiment (5).

### DESCRIPTION OF THE EMBODIMENTS

### Examples

Embodiments of the present invention will be described in detail below with reference to the accompanying drawings.

### 1. First Embodiment

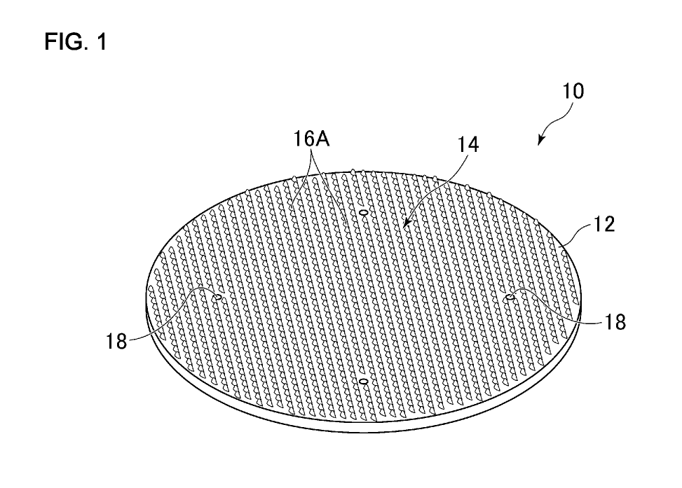

A puncture device 10 shown in FIG. 1 includes a basal portion 12 and a puncture portion 14. The puncture device 10 is formed from a synthetic resin, for example, a thermoplastic resin. The basal portion 12 shown in FIG. 1 has a discoid shape. The puncture portion 14 is disposed on one (first surface) of two surfaces of the basal portion 12.

The puncture device 10 may include channels 18, which guide a drug. In this embodiment, the channel 18 is a hole penetrating the basal portion 12 in the thickness direction of the basal portion 12.

The puncture portion 14 includes a plurality of blade portions 16A. A metal film may be formed on the surface of the puncture portion 14 to improve its mechanical strength.

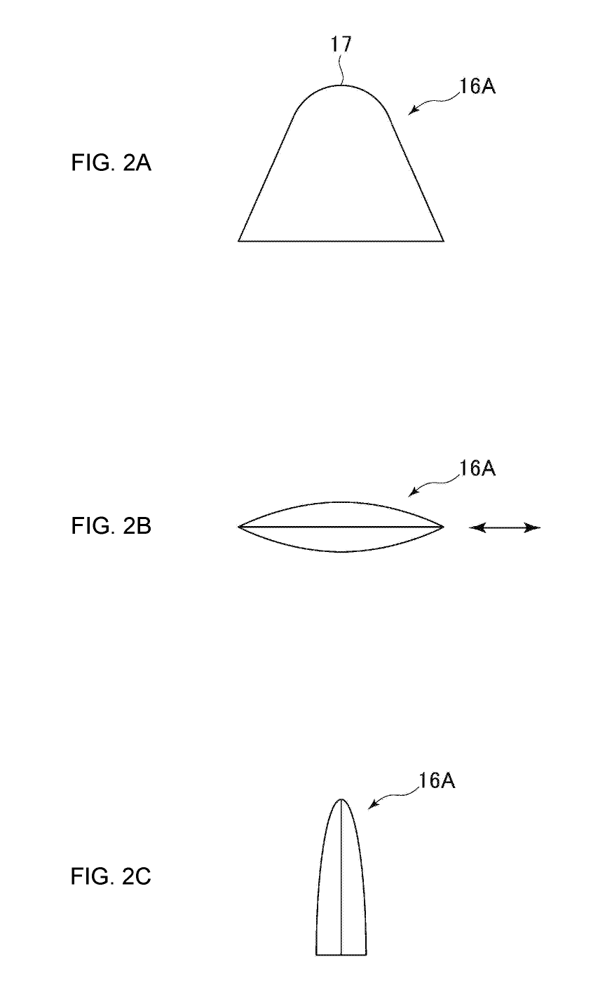

As shown in FIGS. 2A to 2C, the blade portion 16A has a thin shape, and its distal end portion 17 has a convex shape when viewed from the front. In this embodiment, when viewed from the front, the distal end portion 17 has a gentle curve, and the blade portion 16A gradually extends in the widthwise direction toward the proximal end, and thus, the blade portion 16A as a whole has a convex shape like a shark tooth. The blade portion 16A has a substantially elliptical shape when viewed from above. When viewed from the side, the proximal end of the blade portion 16A has a certain thickness, and the blade portion 16A tapers from the proximal end to the distal end. When viewed from the side, the blade portion 16A shown in FIG. 2C has a shape similar to the sides of shells of a clam.

The blade portions 16A having the above-described configuration are arranged on the basal portion 12 with the distal end portions 17 aligned in the same direction (that is, along an arrow direction in FIG. 2B). Thus, the blade portions 16A constitute the puncture portion 14 (FIG. 1).

The puncture device 10 is produced by typical injection molding. A mold (not shown) is preferably formed by combining a plurality of molds in accordance with the shape of the puncture device 10. For example, the blade portion 16A is formed by a mold that can be divided into two parts in the thickness direction. A molten thermoplastic resin is injected into the mold and held by the mold for a predetermined period of time. Thereafter, the mold is opened. Thus, the puncture device 10 is produced.

The other surface (second surface) of the basal portion 12 of the puncture device 10 having the above configuration is attached to a device (not shown) which holds a drug. The puncture portion 14 is brought into contact with the skin and slid in one direction, e.g. a lateral direction (the arrow direction in FIG. 2B) while the puncture portion 14 is pressed against the skin. Thus, the puncture portion 14 punctures a horny layer of the skin. Thereby, the drug guided from the second surface to the first surface of the basal portion 12 through the channels 18 in the basal portion 12 is percutaneously administered through cuts in the skin cut by the puncture portion 14.

In this embodiment, the blade portion 16A included in the puncture portion 14 has a thin shape, and the distal end portion 17 has a convex shape when viewed from the front. Hence the blade portion 16A does not easily deform as compared with a conventional needle-shape, and easily goes into the skin. This allows the blade portion 16A to cut the skin when a force is applied to the blade portion in one direction while the blade portion 16A is pressed against the skin. Therefore, with the puncture portion 14 including the plurality of blade portions 16A, which cut the skin, the puncture device 10 punctures the skin more reliably.

When viewed from the side, the proximal end of the blade portions 16A has the certain thickness and the blade portion 16A tapers from the proximal end to the distal end. This further suppresses the deformation of the distal end when the blade portion 16A is pressed against the skin.

### 2. Second Embodiment

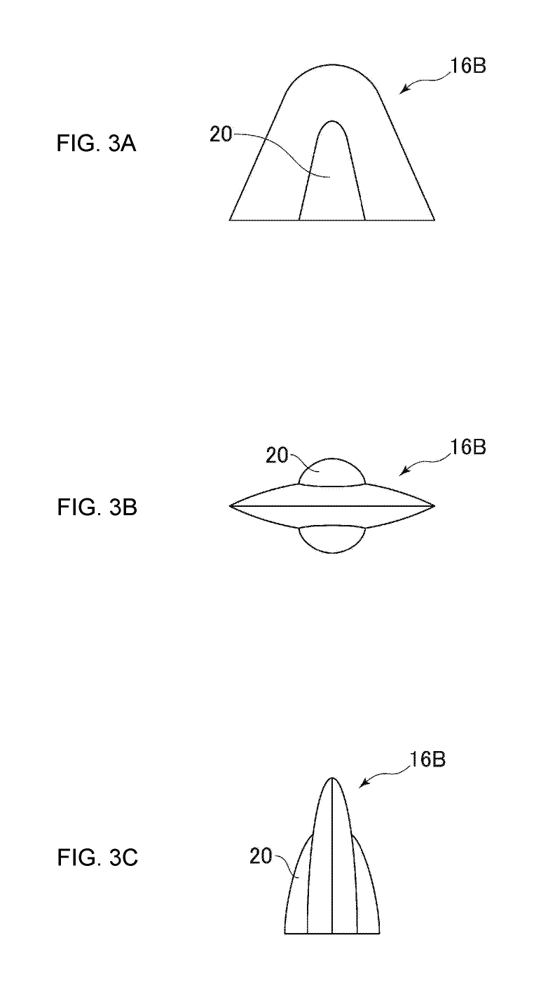

Blade portions according to a second embodiment will be described next. The blade portion according to this embodiment differs from the blade portion according to the first embodiment in that a drug permeation portion is formed. The blade portion according to the second embodiment will be described with reference to FIGS. 3A to 3C, with the same reference numerals denoting the same components as in FIGS. 2A to 2C.

A blade portion 16B shown in FIGS. 3A to 3C has a thin shape, and includes the distal end portion 17 having a convex shape, when viewed from the front, and widened portions 20. The widened portion 20 corresponds to a drug permeation portion. The widened portion 20 includes a protrusion extending linearly from the proximal end of the blade portion 16B toward the distal end of the blade portion 16B. The widened portion 20 is disposed approximately in the middle, when viewed from the front, of each of first and second surfaces of the blade portion 16B.

The blade portion 16B has the thin shape, and the distal end portion 17 has the convex shape when viewed from the front. Hence, effects similar to those of the first embodiment are achieved. In addition, when the blade portion 16B cuts the skin, the widened portions 20 widen the cut made in the skin. A drug permeates the skin through the cut widened by the blade portion 16B and the widened portions 20. Thereby, a drug is administered more efficiently.

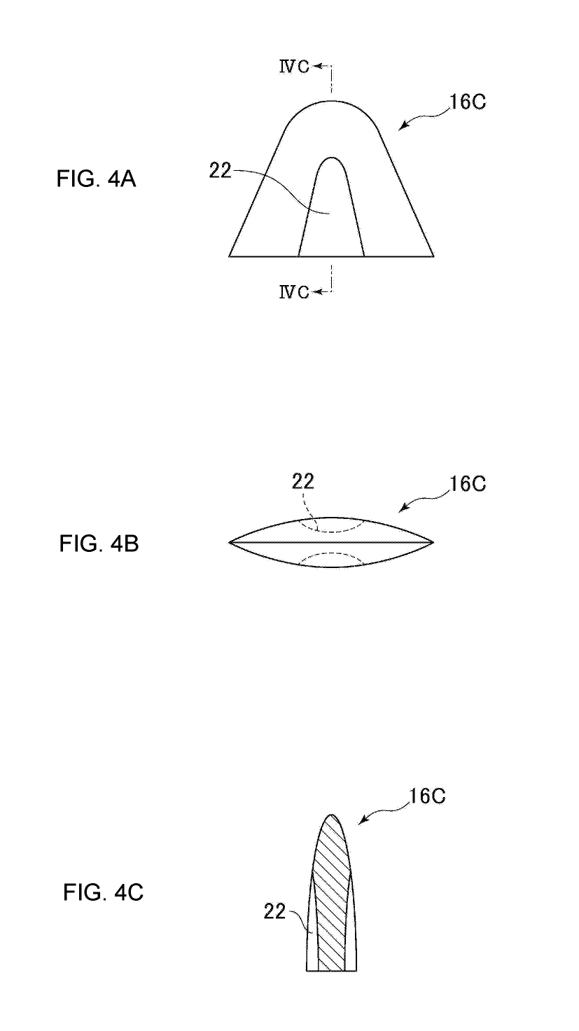

The drug permeation portion is not limited to the widened portion 20 and may include a groove 22 as shown in, for example, FIGS. 4A to 4C. As shown in FIGS. 4A to 4C, the groove 22 extends linearly from the proximal end of a blade portion 16C toward the distal end the blade portion 16C. The groove 22 is disposed approximately in the middle, when viewed from the front, of each of first and second surfaces of the blade portion 16C. When the blade portion 16C cuts the skin, the groove 22 forms a gap between the cut of the skin and the blade portion 16C. A drug permeates the skin through the gap. Thereby, the drug is administered more efficiently.

Channels may be formed through the basal portion at the proximal ends of the grooves 22 of the blade portion 16C. The channel is preferably a hole extending through the basal portion in the thickness direction. Hence, when the blade portion 16C cuts the skin, the drug, which is guided from the second surface to the first surface of the basal portion through the channel, is guided through the groove 22 to the cut in the skin. Thereby, the drug is administered more efficiently.

### 3. Third Embodiment

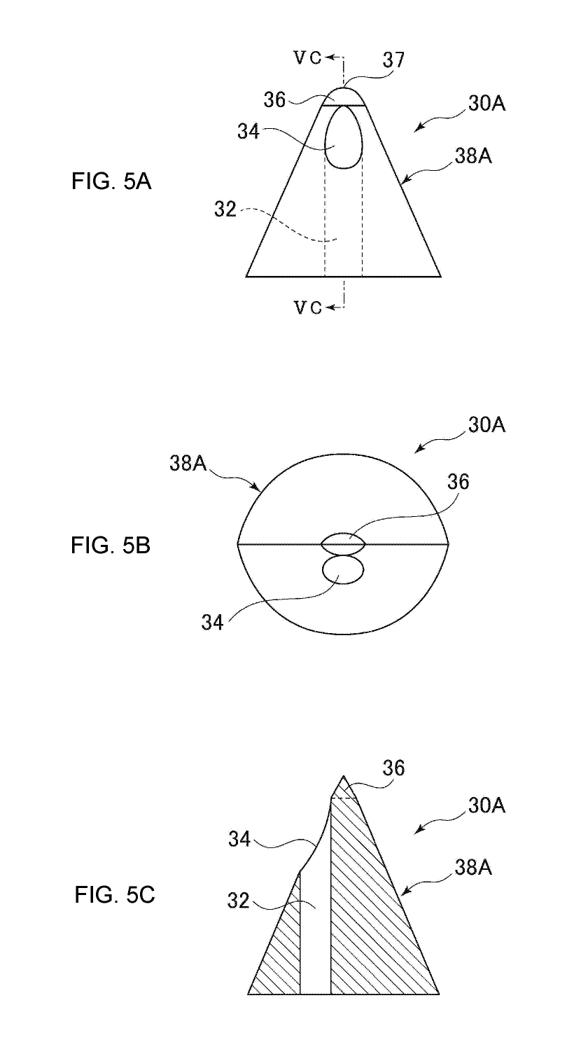

In this embodiment, the blade portion corresponds to a blade edge of a bladed drug supply member including a support portion integrally provided with the proximal end of the blade portion. A plurality of bladed drug supply members constitute a puncture portion of a puncture device. In this puncture device, the blade portion is fixed to the basal portion through the support portion. The bladed drug supply member having the blade portion, being the blade edge, will be described below with reference to FIGS. 5A to 5C.

The puncture device is attached to a syringe or the like and used like an injector, to supply a drug solution. The puncture device has a through hole, which supplies a drug solution. As shown in FIGS. 5A to 5C, a bladed drug supply member 30A has a support portion 38A integrally provided with the proximal end of a blade portion 36. The blade portion 36, being the blade edge, of the bladed drug supply member 30A cuts the skin to make a cut.

The blade portion 36 has substantially the same configuration as that of the blade portion 16A according to the first embodiment. The blade portion 36 has a thin shape, and a distal end portion 37 has a convex shape when viewed from the front. The blade portion 36 has a substantially elliptical shape when viewed from above. When viewed from the front, the distal end portion 37 has a gentle curve, and the blade portion 36 gradually extends in the widthwise direction toward the proximal end, and the blade portion 36 as a whole has a convex shape like a shark tooth. In this regard, the blade portion 36 is substantially the same as the blade portion 16A according to the first embodiment. In the third embodiment, however, the blade portion 36 constitutes the blade edge of the bladed drug supply member 30A, and hence is smaller in size than the blade portion 16A (FIGS. 2A to 2C) in the first embodiment. The bladed drug supply member 30A, as a whole, may have the same shape and the same size as the blade portion 16A when viewed from the front. As will be described below, since the support portion 38A is required to have a certain thickness, the bladed drug supply member 30A is thicker than the blade portion 16A.

The support portion 38A, which is disposed between the basal portion (not shown) and the blade portion 36, supports the blade portion 36. As will be described below, the support portion 38A supplies the drug, which is guided through the basal portion, to the blade portion 36 or the cut. In this embodiment, the support portion 38A has an isosceles trapezoidal shape when viewed from the front. Since the support portion 38A is integrally provided with the proximal end of the blade portion 36, the upper end of the support portion 38A coincides with the proximal end of the blade portion 36. A through hole 32 is provided inside the support portion 38A, and hence the support portion 38A is required to have a certain thickness. In this embodiment, the support portion 38A has a substantially circular-shaped bottom surface when viewed from above.

The through hole 32 is formed inside the support portion 38A to supply a drug from the channel of the basal portion to the blade portion 36. One end of the through hole 32 communicates with the channel. The other end of the through hole 32 reaches the blade portion 36. Hence, the drug is directly guided to the blade portion 36 from the second surface of the basal portion through the channel of the basal portion and the through hole 32 of the support portion 38A. The transverse sectional shape of the through hole 32 is not specifically limited. In this embodiment, the transverse sectional shape of the through hole 32 is approximately circular.

The support portion 38A is not limited to any specific shape and may take any shape as long as the blade portion 36 has the thin shape and the blade portion 36 including the convex-shaped distal end portion 37 is integrally provided with the upper end of the support portion 38A and the through hole 32, which guides a drug to the blade portion 36, is formed inside the support portion 38A. The bladed drug supply members 30A constitute the puncture portion of the puncture device. Each of the bladed drug supply members 30A includes the support portion 38A, which is integrally provided with the lower end of the blade portion 36, and the blade portion 36, which corresponds to the blade edge.

The puncture device including the plurality of bladed drug supply members 30A as the puncture portion is produced by typical injection molding as in the first embodiment. A mold (not shown) is preferably formed by combining a plurality of molds so as to produce the support portion 38A integrally with the blade portion 36 as described above. For example, the bladed drug supply member 30A is formed by a mold which includes a rod for forming the through hole 32 inside the support portion 38A and which can be divided into two parts in the thickness direction. The puncture device according to this embodiment is produced in the same manner as in the first embodiment except for the use of the predetermined mold.

In this embodiment, the blade portion 36 has a thin shape, and the distal end portion 37 has a convex shape when viewed from the front. Hence, the effects similar to those of the first embodiment are achieved. The through hole 32 inside the support portion 38A of the bladed drug supply member 30A is provided to communicate with the channel of the basal portion (not shown). Since the support portion 38A includes the through hole 32 communicating with the channel, when the blade portion 36 cuts the skin, a drug is guided from the second surface of the basal portion into the through hole 32 of the support portion 38A through the channel. The drug is supplied from an opening end 34 of the through hole 32 to the blade portion 36, being the blade edge, and permeates the skin through the cut. Thereby, the drug is supplied close to the blade portion 36, so that the drug is administered more efficiently.

After the blade portion 36 cuts the skin, the puncture device is pushed to insert at least a part of the opening end 34 of the through hole 32 of the bladed drug supply member 30A into the cut. Thereby, the drug is administered. In this case, the drug guided from the second surface of the basal portion through the channel is directly supplied from the opening end 34 into the cut. Thereby, the drug is administered more efficiently.

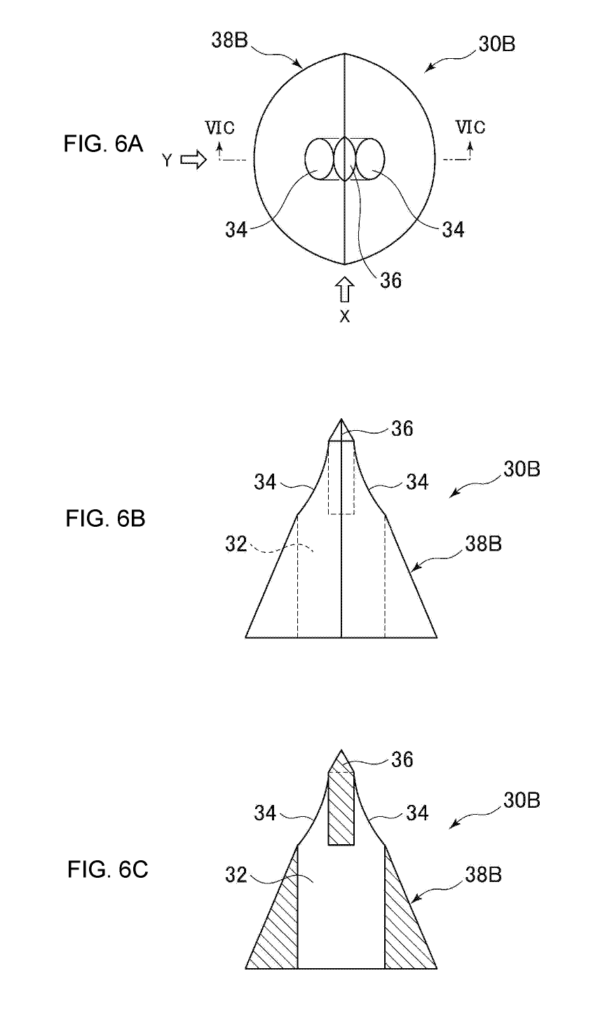

The number of the opening end 34 of the support portion 38A is not limited to one and the support portion 38A may be provided with a plurality of opening ends. For example, as shown in FIG. 6A, the opening ends 34 may be respectively provided on the first and second surfaces of a support portion 38B. The blade portion 36 is disposed between the opening ends 34. In a bladed drug supply member 30B, the support portion 38B is integrally provided with the proximal end of the blade portion 36. FIG. 6B is a view of the bladed drug supply member 30B including the support portion 38B, taken along an arrow X. Although not shown, a view of the bladed drug supply member 30A taken along an arrow Y is similar to the front view shown in FIG. 5A. The two opening ends 34 of the bladed drug supply member 30B are the opening ends of the single through hole 32 as shown in FIG. 6C, which is the cross-sectional view cut along a line VIC-VIC. The two opening ends may be the opening ends of different through holes. The two opening ends 34 increase the amount of drug supplied to the blade portion 36 through the through hole 32. Hence, the efficiency of administration is further improved.

### 4. Modified Embodiment

The present invention is not limited to the above embodiments, and may be changed within the scope of the present invention.

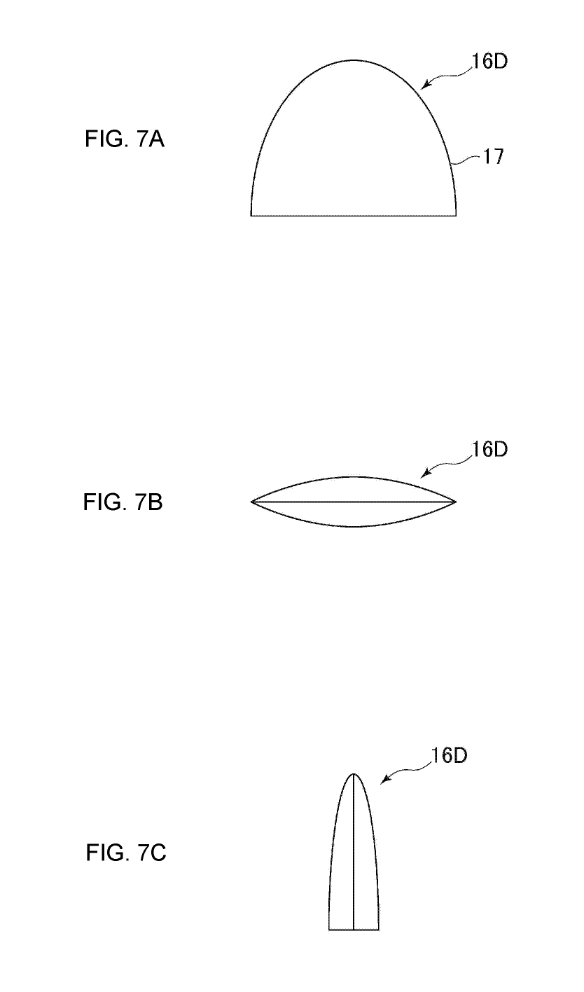

For example, as shown in FIGS. 7A to 7C, a blade portion 16D having a substantially semicircular shape, when viewed from the front, may be used. In this case, the blade portion 16D has a thin shape, and the distal end portion 17 has a convex shape when viewed from the front. Hence, effects similar to those of the above-described first embodiment are achieved.

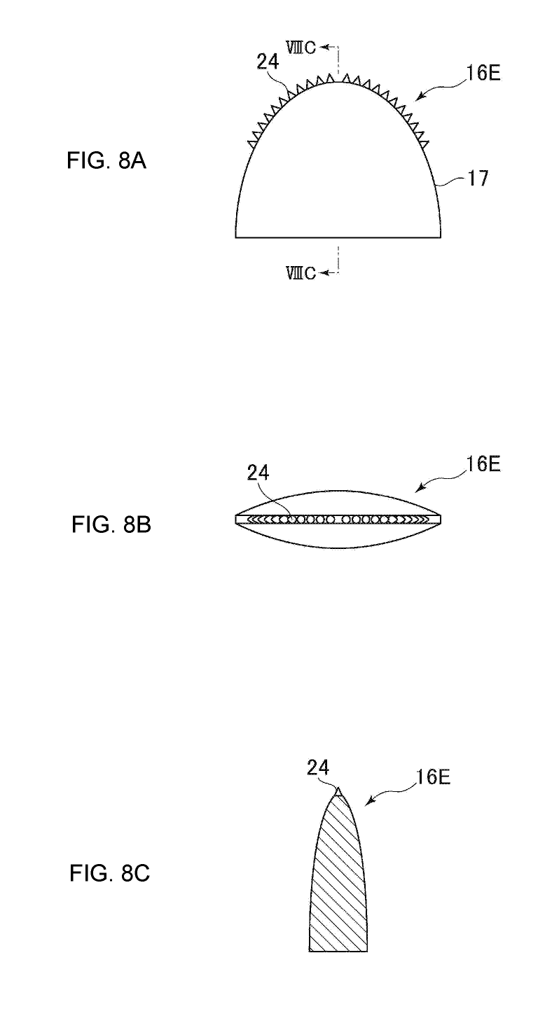

As shown in FIGS. 8A to 8C, a blade portion 16E may include teeth (serrations) 24 on the distal end portion 17. The blade portion 16E with the teeth 24 cuts the skin more efficiently.

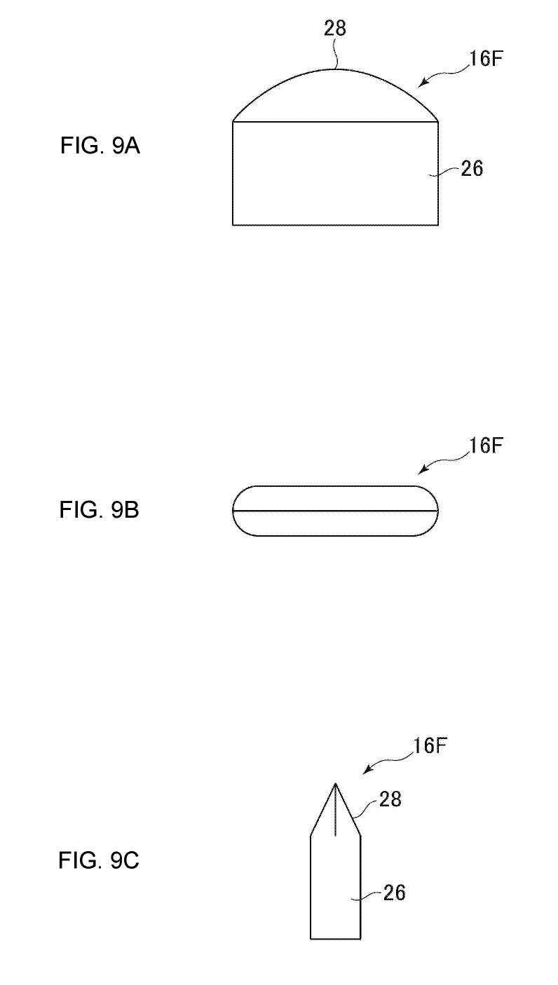

As shown in FIGS. 9A to 9C, a blade portion 16F may include a plate-shape proximal end portion 26 and a distal end portion 28 having a convex shape. The blade portion 16F has an oval shape when viewed from above. With the blade portion 16F having the thin shape and the distal end portion 28 having the convex shape when viewed from the front, effects similar to those of the first embodiment are achieved. The plate-shaped proximal end portion 26 improves the mechanical strength of the blade portion 16F to allow the formation of the sharper distal end portion 28. The blade portion 16F therefore cuts the skin more efficiently.

Each of the blade portions 16D to 16F according to the above modified embodiments may be provided with the drug permeation portion described in the second embodiment. Each of the blade portions 16D to 16F according to the modified embodiments may have an appropriate size, and the support portion described in the third embodiment may be integrally provided with the lower end of the blade portion, to form a bladed drug supply member. The puncture portion may be formed by combining various types of the blade portions according to the above embodiments and the modified embodiments.

In the above embodiments, the blade portions have the same size. The present invention is not limited to this. The blade portions located in a center portion of the puncture portion may protrude higher in the direction toward their distal ends than those in a peripheral portion of the puncture portion. The puncture portion having such a shape is produced by making a center portion of the basal portion protrude toward the distal ends of the blade portions. Thereby, the entire puncture portion of the puncture device makes the cuts having substantially the same depth in the skin.

The above embodiments describe the puncture device formed from a synthetic resin. The present invention is not limited to this. The puncture device may be formed from a polylactic acid biodegradable polymer or a metal such as stainless steel, titanium, silver, or gold. As in the case in which a synthetic resin is used, in a case where a puncture device is formed from a polylactic acid biodegradable polymer, a metal film may be provided on the surface of the blade portion to increase the mechanical strength of the blade portion. When a puncture device is formed from metals, the puncture device may be formed from a multilayer film, which is a stack of films made of different metals. A surface of the multilayer metal film is provided with plating having a higher strength, to further increase the mechanical strength.

The plating is effective in increasing the strength of a puncture device made of a metal, such as stainless steel, or a synthetic resin. The plating may be selected from, for example, bright chromium, velour chromium, bright nickel, velour nickel, bright tin-cobalt, velour tin-cobalt, bright gold, velour gold, trivalent chromium, bright Noburoi, velour Noburoi, rhodium plating, and platinum plating.

The above embodiments describe the channels, which are the holes formed in the basal portion in the thickness direction. The present invention is not limited to this. The channels in the first and second embodiments may be grooves formed along the sides of the basal portion.

In addition to the case in which the drug is percutaneously administered by being guided from the second surface to the first surface of the basal portion through the channels formed in the basal portion, the above embodiments describe the case in which the drug is percutaneously administered through the cut by being guided to the blade portion through the channel formed in the basal portion and the through hole provided in the support portion of the bladed drug supply member or by being directly supplied to the cut from the opening end of the through hole provided in the support portion. The present invention is not limited to them. For example, the blade portion may be coated with a drug in advance, and the drug may be percutaneously administered through the cut at the same time as the blade portion cuts the skin.

The above embodiments describe that the basal portion has the discoid shape. The present invention is not limited to this. The basal portion may be a triangular or rectangular plate-shaped member.

The above embodiments describe that the distal end portions of the blade portions have the gently curved shapes when viewed from the front. The present invention is not limited to this. The distal end portion of the blade portion may have a pointed shape when viewed from the front.

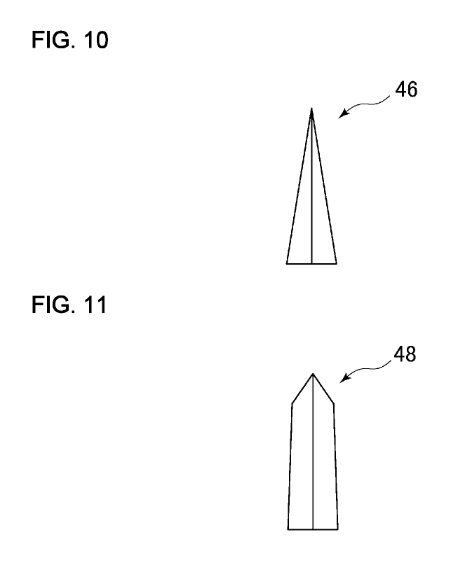

The above embodiments describe that the blade portions have the shapes similar to the sides of the shells of a clam, when viewed from the side. The present invention is not limited to this. As shown in FIG. 10, a blade portion 46 may have a substantially isosceles triangle shape when viewed from the side. As shown in FIG. 11, a blade portion 48 may have a shape in which a thick proximal end is connected linearly to a tapered distal end, when viewed from the side.

In the third embodiment, the thicknesses of the support portions 38A and 38B of the bladed drug solution supply members 30A and 30B are not specifically limited as long as the through holes 32 are formed inside.

The blade portions 36 may be provided not only on the distal end portions 37 of the bladed drug solution supply members 30A and 30B but also on the entire outer edges of the bladed drug solution supply members 30A and 30B.

The third embodiment may be combined with the second embodiment. For example, grooves 22 like those shown in FIG. 4 may be provided on the first and second surfaces of the bladed drug supply member. The through hole 32 may be provided with its opening inside the groove 22. In this case, the groove 22 may be provided from a height approximately ⅓ of the height of the bladed drug solution supply member 30A or 30B from the proximal end of the bladed drug solution supply member 30A or 30B. The angle of the distal end may be set to approximately 15° when viewed from the side. The groove 22 is not limited to a straight shape and may be angled at an angle of approximately 6°.

### REFERENCE NUMERALS

10 puncture device

12 basal portion

14 puncture portion

16A to 16F, 36, 46, 48 blade portion

17, 28, 37 distal end portion

18 channel

20 widened portion (drug permeation portion)

22 groove (drug permeation portion)

24 teeth

26 proximal end portion

38A, 38B support portion

30A, 30B bladed drug supply member

32 through hole

34 opening end

1. A puncture device comprising:
a basal portion; and
a plurality of blade portions disposed on the basal portion, the blade portion having a thin shape, a distal end portion of the blade portion having a convex shape.

2. The puncture device according to claim 1, wherein teeth are disposed on the distal end portion.

3. The puncture device according to claim 1, wherein the blade portion includes a drug permeation portion.

4. The puncture device according to claim 3, wherein the drug permeation portion includes a widened portion, the widened portion including a protrusion linearly extending from a proximal end of the blade portion toward a distal end of the blade portion.

5. The puncture device according to claim 3, wherein the drug permeation portion includes a groove linearly extending from a proximal end of the blade portion toward a distal end of the blade portion.

6. The puncture device according to claim 1, wherein a center portion of the basal portion protrudes toward a distal end of the blade portion.

7. The puncture device according to claim 1, wherein the basal portion includes a channel guiding a drug to a first surface of the basal portion from a second surface of the basal portion, the blade portion being disposed on the first surface.

8. The puncture device according to claim 7, wherein the blade portion is fixed to the basal portion through a support portion integrally provided with the proximal end of the blade portion, and the support portion includes a through hole communicating with the channel.

9. The puncture device according to claim 1, wherein the blade portion is formed from a synthetic resin, polylactic acid biodegradable polymer, or metal.

10. The puncture device according to claim 9, wherein a surface of the blade portion is provided with a metal film which reinforces a mechanical strength of the blade portion.
